(19) United States
(12) Patent Application Publication
(10) Pub. No.: US 2005/0261668 A1
(43) Pub. Date: Nov. 24, 2005

(54) Drug delivery device

(75) Inventors: Linda Mosack, Webster, NY (US); Daniel J. Hook, Walworth, NY (US); George L. Grobe III, Pittsford, NY (US); Matthew Jonasse, Sodus, NY (US)

Correspondence Address: Bausch & Lomb Incorporated, One Bausch & Lomb Place, Rochester, NY, 14604-2701, US

(73) Assignee: Bausch & Lomb Incorporated, Rochester, NY (US)

(21) Appl. No.: 10/403,421

(22) Filed: Mar. 28, 2003

(51) Int. Cl. A61K 9/24; A61K 9/22

(52) U.S. Cl. 604/890.1; 424/471

(57) ABSTRACT

A drug delivery device includes a drug core having a pharmaceutically active agent, a holder that holds the drug core and is made of a material impermeable to passage of the active agent; and a tab for attaching the device to eye tissue. A surface of the holder is plasma treated and adhered to at least one of the drug core and the tab. The device is preferably designed for placing in the eye, such as implanting the device using the tab, so the active agent is released in a sustained manner to the eye.

### FIELD OF THE INVENTION

This invention relates to a drug delivery device, preferably a device that is placed or implanted in the eye to release a pharmaceutically active agent to the eye. At least one surface of the device is plasma treated to improve adherence between that surface and another surface of the device.

### BACKGROUND OF THE INVENTION

Various drugs have been developed to assist in the treatment of a wide variety of ailments and diseases. However, in many instances, such drugs cannot be effectively administered orally or intravenously without the risk of detrimental side effects. Additionally, it is often desired to administer a drug locally, i.e., to the area of the body requiring treatment. Further, it may be desired to administer a drug locally in a sustained release manner, so that relatively small doses of the drug are exposed to the area of the body requiring treatment over an extended period of time.

Accordingly, various sustained release drug delivery devices have been proposed for placing in the eye and treating various eye diseases. Examples are found in the following patents, the disclosures of which are incorporated herein by reference: US 2002/0086051A1 (Viscasillas); US 2002/0106395A1 (Brubaker); US 2002/0110591A1 (Brubaker et al.); US 2002/0110592A1 (Brubaker et al.); US 2002/0110635A1 (Brubaker et al.); U.S. Pat. No. 5,378,475 (Smith et al.); U.S. Pat. No. 5,773,019 (Ashton et al.); U.S. Pat. No. 5,902,598 (Chen et al.); U.S. Pat. No. 6,001,386 (Ashton et al.); U.S. Pat. No. 6,217,895 (Guo et al.); and U.S. Pat. No. 6,375,972 (Guo et al.).

Many of these devices include an inner drug core including a pharmaceutically active agent, and some type of holder for the drug core made of an impermeable material such as silicone or other hydrophobic materials. This impermeable holder may be adhered to a different material, such as polyvinyl alcohol (PVA) or other hydrophobic material.

The present invention recognized that in such devices, problems may arise in adhering surfaces of the hydrophobic and hydrophilic materials to one another. For ophthalmic sustained release drug delivery devices, it is critical that the devices do not lose their integrity while implanted in the eye. If the two different materials do not adhere together adequately, the device could lose its structural integrity after implantation. The present invention addresses this problem by providing devices with more reliable structural integrity, and therefore additional safety. Additionally, this invention provides methods of making such devices which can be more easily and reliably reproduced on a commercial manufacturing scale.

### BRIEF DESCRIPTION OF THE DRAWINGS

FIG. 1 is a perspective schematic view of a first embodiment of a drug delivery device of this invention.

FIG. 2 is a cross-sectional view of the device of FIG. 1.

FIG. 3 is a cross-sectional schematic view of a second embodiment of a drug delivery device.

FIG. 4 is a cross-sectional schematic view of a third embodiment of a drug delivery device.

### SUMMARY OF THE INVENTION

This invention provides a drug delivery device. According to various embodiments, the device comprises: a drug core comprising a pharmaceutically active agent; a holder that holds the drug core, the holder being made of a material impermeable to passage of the active agent; and a tab for attaching the device to eye tissue, wherein a surface of the holder is plasma treated and adhered to at least one of the drug core and the tab. The device is preferably designed for placing in the eye, such as implanting the device using the tab, so the active agent is released in a sustained manner to the eye.

This invention also provides methods of making such devices. According to various embodiments, the method comprises: providing a holder made of a material impermeable to passage of a pharmaceutically active agent and plasma treating a surface of the holder; inserting in the holder a drug core comprising the active agent; and adhering to the plasma treated surface a tab for attaching the device to eye tissue. The methods of the invention also include: providing a drug core comprising a pharmaceutically active agent; providing a holder made of a material impermeable to passage of the active and plasma treating a surface of the holder; and inserting the drug core in the holder, and adhering the drug core to the plasma treated surface of the holder.

The invention provides a method of using such a device, comprising: providing a drug delivery device, comprising a drug core comprising a pharmaceutically active agent; a holder that holds the drug core, the holder being made of a material impermeable to passage of the active agent, and a tab for attaching the device to eye tissue, wherein a surface of the holder is plasma treated and adhered to at least one of the drug core and the tab; and attaching the tab to eye tissue.

### DETAILED DESCRIPTION OF PREFERRED EMBODIMENTS

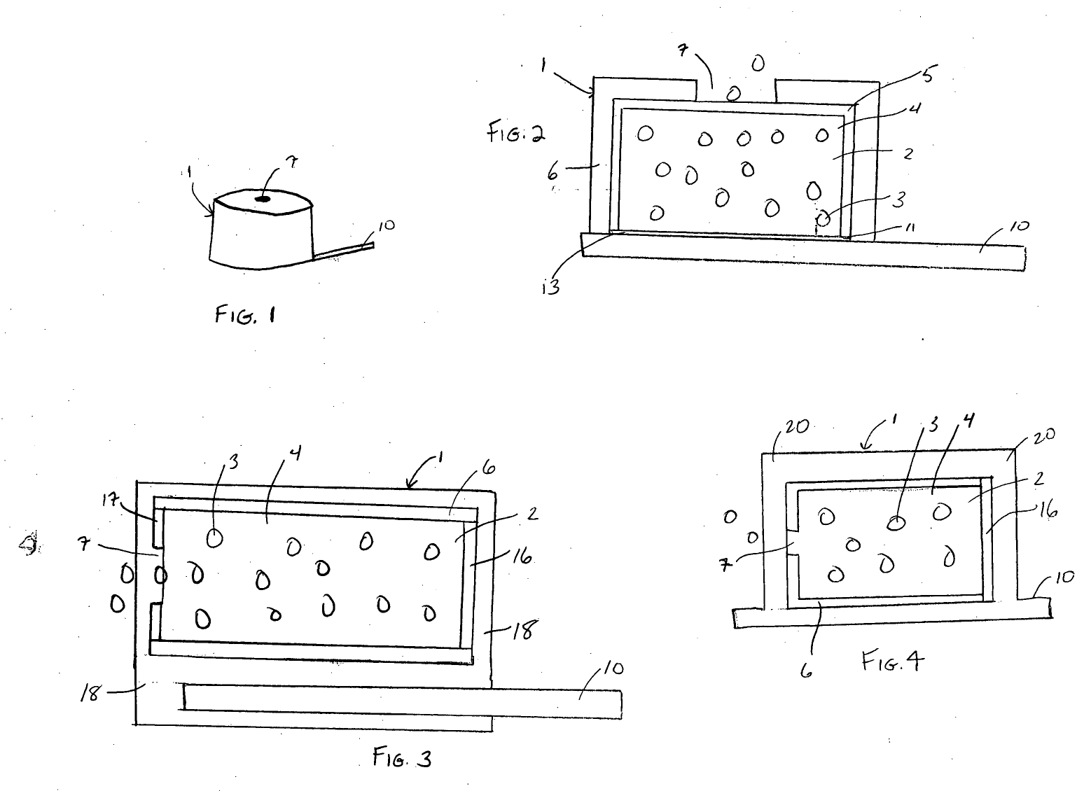

FIGS. 1 and 2 illustrate a first embodiment of a device of this invention. Device 1 is a sustained release drug delivery device for implanting in the eye. Device 1 includes inner drug core 2 including a pharmaceutically active agent 3.

This active agent may include any compound, composition of matter, or mixture thereof that can be delivered from the device to produce a beneficial and useful result to the eye, especially an agent effective in obtaining a desired local or systemic physiological or pharmacological effect. Examples of such agents include: anesthetics and pain killing agents such as lidocaine and related compounds and benzodiazepam and related compounds; anti-cancer agents such as 5-fluorouracil, adriamycin and related compounds; anti-fungal agents such as fluconazole and related compounds; anti-viral agents such as trisodium phosphomonoformate, trifluorothymidine, acyclovir, ganciclovir, DDI and AZT; cell transport/mobility impending agents such as colchicine, vincristine, cytochalasin B and related compounds; antiglaucoma drugs such as beta-blockers: timolol, betaxolol, atenalol, etc; antihypertensives; decongestants such as phenylephrine, naphazoline, and tetrahydrazoline; immunological response modifiers such as muramyl dipeptide and related compounds; peptides and proteins such as cyclosporin, insulin, growth hormones, insulin related growth factor, heat shock proteins and related compounds; steroidal compounds such as dexamethasone, prednisolone and related compounds; low solubility steroids such as fluocinolone acetonide and related compounds; carbonic anhydrize inhibitors; diagnostic agents; antiapoptosis agents; gene therapy agents; sequestering agents; reductants such as glutathione; antipermeability agents; antisense compounds; antiproliferative agents; antibody conjugates; antidepressants; bloodflow enhancers; antiasthmatic drugs; antiparasiticagents; non-steroidal anti inflammatory agents such as ibuprofen; nutrients and vitamins: enzyme inhibitors: antioxidants; anticataract drugs; aldose reductase inhibitors; cytoprotectants; cytokines, cytokine inhibitors. and cytokin protectants; uv blockers; mast cell stabilizers; and anti neovascular agents such as antiangiogenic agents like matrix metalloprotease inhibitors.

Examples of such agents also include: neuroprotectants such as nimodipine and related compounds; antibiotics such as tetracycline, chlortetracycline, bacitracin, neomycin, polymyxin, gramicidin, oxytetracycline, chloramphenicol, gentamycin, and erythromycin; antiinfectives; antibacterials such as sulfonamides, sulfacetamide, sulfamethizole, sulfisoxazole; nitro furazone, and sodium propionate; antiallergenics such as antazoline, methapyriline, chlorpheniramine, pyrilamine and prophenpyridamine; anti-inflammatories such as hydrocortisone, hydrocortisone acetate, dexamethasone 21-phosphate, fluocinolone, medrysone, methyiprednisolone, prednisolone 21-phosphate, prednisolone acetate, fluoromethalone, betamethasone and triminolone; miotics and anti-cholinesterase such as pilocarpine, eserine salicylate, carbachol, di-isopropyl fluorophosphate, phospholine iodine, and demecarium bromide; mydriatics such as atropine sulfate, cyclopentolate, homatropine, scopolamine, tropicamide, eucatropine, and hydroxyamphetamine; svmpathomimetics such as epinephrine; and prodrugs such as those described in Design of Prodrugs, edited by Hans Bundgaard, Elsevier Scientific Publishing Co., Amsterdam, 1985. In addition to the above agents, other agents suitable for treating, managing, or diagnosing conditions in a mammalian organism may be placed in the inner core and administered using the sustained release drug delivery devices of the current invention. Once again, reference may be made to any standard pharmaceutical textbook such as Remington's Pharmaceutical Sciences for the identity of other agents.

Any pharmaceutically acceptable form of such a compound may be employed in the practice of the present invention, i.e., the free base or a pharmaceutically acceptable salt or ester thereof. Pharmaceutically acceptable salts, for instance, include sulfate, lactate, acetate, stearate, hydrochloride, tartrate, maleate and the like.

For the illustrated embodiment, the active agent employed is fluocininolone acetonide.

As shown in the illustrated embodiment, active agent 3 may be mixed with a matrix material 4. Preferably, matrix material 4 is a polymeric material that is compatible with body fluids and the eye. Additionally, matrix material should be permeable to passage of the active agent 3 therethrough, particularly when the device is exposed to body fluids. For the illustrated embodiment, the matrix material is PVA. Also, in this embodiment, inner drug core 2 may be coated with a coating 5 of additional matrix material which may be the same or different from material 4 mixed with the active agent. For the illustrated embodiment, the coating 5 employed is also PVA.

Device 1 includes a holder 6 for the inner drug core 2. Holder 6 is made of a material that is impermeable to passage of the active agent 3 therethrough. Since holder 6 is made of the impermeable material, a passageway 7 is formed in holder 6 to permit active agent 3 to pass therethrough and contact eye tissue. In other words, active agent passes through any permeable matrix material 4 and permeable coating 5, and exits the device through passageway 7. For the illustrated embodiment, the holder is made of silicone, especially polydimethylsiloxane (PDMS) material.

A wide variety of materials may be used to construct the devices of the present invention. The only requirements are that they are inert; non-immunogenic and of the desired permeability. Materials that may be suitable for fabricating the device include naturally occurring or synthetic materials that are biologically compatible with body fluids and body tissues, and essentially insoluble in the body fluids with which the material will come in contact. The use of rapidly dissolving materials or materials highly soluble in body fluids are to be avoided since dissolution of the wall would affect the constancy of the drug release, as well as the capability of the device to remain in place for a prolonged period of time.

Naturally occurring or synthetic materials that are biologically compatible with body fluids and eye tissues and essentially insoluble in body fluids which the material will come in contact include, but are not limited to, glass, metal, ceramics, polyvinyl acetate, cross-linked polyvinyl alcohol, cross-linked polyvinyl butyrate, ethylene ethylacrylate copolymer, polyethyl hexylacrylate, polyvinyl chloride, polyvinyl acetals, plasiticized ethylene vinylacetate copolymer, polyvinyl alcohol, polyvinyl acetate, ethylene vinylchloride copolymer, polyvinyl esters, polyvinylbutyrate, polyvinylformal, polyamides, polymethylmethacrylate, polybutylmethacrylate, plasticized polyvinyl chloride, plasticized nylon, plasticized soft nylon, plasticized polyethylene terephthalate, natural rubber, polyisoprene, polyisobutylene, polybutadiene, polyethylene, polytetrafluoroethylene, polyvinylidene chloride, polyacrylonitrile, cross-linked polyvinylpyrrolidone, polytrifluorochloroethylene, chlorinated polyethylene, poly(1,4′-isopropylidene diphenylene carbonate), vinylidene chloride, acrylonitrile copolymer, vinyl chloride-diethyl fumerale copolymer, butadiene/styrene copolymers, silicone rubbers, especially the medical grade polydimethylsiloxanes, ethylene-propylene rubber, silicone-carbonate copolymers, vinylidene chloride-vinyl chloride copolymer, vinyl chloride-acrylonitrile copolymer and vinylidene chloride-acrylonitride copolymer.

The illustrated embodiment includes a tab 10 which may be made of a wide variety of materials, including those mentioned above for the matrix material and/or the holder. Tab 10 may be provided in order to attach the device to a desired location in the eye, for example, by suturing. For the illustrated embodiment, tab 10 is made of PVA and is adhered to the inner drug core 2 with adhesive 11. Adhesive 11 may be a curable silicone adhesive, a curable PVA solution, or the like.

The present invention recognized that difficulties often arose when trying to adhere different materials to one another, especially a hydrophilic material such as PVA to a hydrophobic silicone holder. As one example, in some cases the tab may not consistently adhere to the device, meaning the device could lose its structural integrity after being implanted in the eye. Also, the surfaces of the silicone holder tend to non-wettable, and in the case that liquid PVA is added to the silicone holder, the liquid may not evenly wet the surfaces of the holder and/or adequately adhere to the silicone holder when cured.

The present invention solves this problem by increasing the wettability of the holder and improving the adherence of the drug core and/or the tab to the holder.

This is accomplished by plasma treating surfaces of the holder contacting the drug core and/or the tab. Because the device is relatively small, it will generally be practical to plasma treat the entire holder.

Although plasma processes are generally well known in the art, a brief overview is provided. Plasma surface treatments involve passing an electrical discharge through a gas at low pressure. The electrical discharge may be at radio frequency (typically 13.56 MHz), although microwave and other frequencies can be used. Electrical discharges produce ultraviolet (UV) radiation, in addition to being absorbed by atoms and molecules in their gas state, resulting in energetic electrons and ions, atoms (ground and excited states), molecules, and radicals. Thus, a plasma is a complex mixture of atoms and molecules in both ground and excited states, which reach a steady state after the discharge is begun. The circulating electrical field causes these excited atoms and molecules to collide with one another as well as the walls of the chamber and the surface of the material being treated.

Preferably, the holder is oxidized by the use of an oxidation plasma to render surfaces of the holder more wettable and more adherent. Such oxidation may be accomplished in an atmosphere composed of an oxidizing media such as oxygen or nitrogen containing compounds: ammonia, an aminoalkane, air, water, peroxide, oxygen gas, methanol, acetone, alkylamines, and the like, or appropriate mixtures thereof including inert gases such as argon. Examples of mixed media include oxygen/argon or hydrogen/methanol. Typically, the treatment is conducted in a closed chamber at an electric discharge frequency of 13.56 Mhz, preferably between about 20 to 500 watts at a pressure of about 0.1 to 1.0 torr, preferably for about 10 seconds to about 10 minutes or more, more preferably about 1 to 10 minutes.

The oxidizing plasma tends to etch the surface of the holder, creating radicals and oxidized functional groups and rendering the surface more wettable and more adherent. This surface treatment is often referred to as plasma oxidation, electrical glow discharge, corona discharge, or like terms, and as used herein, the term “plasma treat” encompasses all such methods. Experiments have shown that such plasma treatment of silicone holders results in the surfaces of the holder having a higher concentration of oxygen than prior to treatment.

According to preferred embodiments, the holder is also extracted to remove residual materials therefrom. For example, in the case of silicone, the holder may include lower molecular weight materials such as unreacted monomeric material and oligomers. It is believed that the presence of such residual materials may also deleteriously affect adherence of the holder surfaces. The holder may be extracted by placing the holder in an extraction solvent, optionally with agitation. Representative solvents are polar solvents such as isopropanol, heptane, hexane, toluene, tetrahydrofuran (THF), chloroform, supercritical carbon dioxide, and the like, including mixtures thereof. After extraction, the solvent is preferably removed from the holder, such as by evaporation in a nitrogen box, a laminar flow hood or a vacuum oven. When extraction is used, it is preferably performed prior to the plasma treatment.

A device of the type shown in FIGS. 1 and 2 may be manufactured as follows. First, fluocininolone acetonide, the active agent, is provided in the form of a micronized powder, and then is mixed with an aqueous solution of the matrix material, PVA, whereby the fluocininolone acetonide and PVA agglomerate into larger sized particles. The resulting mixture is then dried to remove some of the moisture, and then milled and sieved to reduce the particle size so that the mixture is more flowable. Optionally, a small amount of inert lubricant, for example, magnesium stearate, may be added to assist in tablet making. This mixture is then formed into a tablet using standard tablet making apparatus.

A cylindrical cup of silicone is separately formed, for example by molding, having a size generally corresponding to the tablet and a shape as generally shown in FIG. 2. This silicone holder is then extracted with a solvent such as isopropanol and plasma treated to oxidize the surface in an environment such as oxygen. If desired, a drop of liquid PVA may be placed into the holder through the open end 13 of the holder. Then, the inner drug core tablet is placed into the silicone holder through the same open end and pressed into the cylindrical holder. If the drop of liquid PVA has been applied, the pressing of the tablet causes the liquid PVA to fill the space between the tablet inner core and the silicone holder, thus forming permeable layer 5 shown in FIGS. 1 and 2. A layer of adhesive is applied to the open side of the holder to fully enclose the inner drug core tablet at this end. Tab 10 is inserted at this end of the device. The liquid PVA and adhesive are cured by heating the assembly.

### EXAMPLE 1

The following experiments were conducted to determine the effect of plasma treatment on adhesive strength between PVA and silicone used in ophthalmic drug delivery devices.

PVA strips were cut from precured PVA film, and then the strips were placed in an oven for three hours at 150° C. to further cure the strips. Silicone strips made of PDMS were provided. The PVA strips simulate a suture tab and the silicone strips simulate a silicone holder. Some of the silicone strips holders were extracted in isopropanol (IPA), while some holders were not extracted. Some of the strips were plasma treated, while some holders were not plasma treated. The plasma treated holders were subjected to three different plasma treatments: treatment in argon; treatment in argon/oxygen mixture; treatment in oxygen. The silicone strips were cut to a size of 1.93×3.81 mm. PVA strips were glued to each side of the silicone holders using a 10% PVA solution, and then left overnight. The assembly was dip coated in PVA three times, with each application allowed to dry for two hours. Finally, the assembly was cured in an oven for five hours at 135° C. Mechanical testing was conducted on individual assemblies by gripping each PVA strip with a clamp, under the following conditions: 6.35 mm/min, 15.95 mm gauge length, and 50,000 g load cell. The results are summarized in Table 1, noting that the reported measurement is the load (g) reached at breakage.

TABLE 1

| Treatment | | | | | | |
|---|---|---|---|---|---|---|
| Plasma | Extraction | Sample 1 | Sample 2 | Sample 3 | Average | Note |
| None | None | — | — | — | — | All broke at clamp grips |
| None | IPA | — | 2097 | 1466 | 1782 | Broke at adhesive bond |
| Ar | IPA | 2585 | 2238 | — | 2412 | Broke at adhesive bond |
| Ar/O₂ | IPA | — | — | 2651 | 2651 | Broke at adhesive bond |
| O₂ | IPA | — | — | — | >3071 | Did not break at 3071 g |

### EXAMPLE 2

The following experiments were conducted to determine the effect of plasma treatment on adhesive strength between PVA and silicone used in ophthalmic drug delivery devices.

PVA strips were cut from precured PVA film, and then the strips were placed in an oven for three hours at 150° C. to further cure the strips. Silicone strips made of PDMS were provided. The PVA strips simulate a suture tab and the silicone strips simulate a silicone holder. Some of the silicone strips holders were extracted in isopropanol (IPA), heptane or supercritical carbon dioxide (CO₂), while some holders were not extracted. Some of the holders were plasma treated in an oxygen environment, while some holders were not plasma treated. The silicone strips were cut to a size of 2×4 mm. PVA strips were glued to each side of the silicone holders using a 10% PVA solution, and then left overnight. Finally, the assembly was cured in an oven for five hours at 135° C. The assemblies were then hydrated in acetate buffered saline solution overnight. Mechanical testing was conducted on individual assemblies by gripping each PVA strip with a clamp, under the following conditions: 6.35 mm/min, 15.95 mm gauge length, and 2,000 g load cell. The results are summarized in Table 2, noting that the reported measurement is the load (g) reached at breakage and ten samples were used for each treatment.

TABLE 2

| Treatment | | Average Breakage | |
|---|---|---|---|
| Plasma | Extraction | (std deviation) | Note |
| None | None | 44.24 (35.06) | 3/10 fell apart during |
| | | | hydration |
| O₂ | None | 89.37 (54.88) | None fell apart during |
| | | | hydration |
| O₂ | IPA | 279.26 (70.92) | None fell apart during |
| | | | hydration |
| O₂ | Heptane | 264.83 (73.19) | None fell apart during |
| | | | hydration |
| O₂ | CO₂ | 259.61 (46.45) | None fell apart during |
| | | | hydration |

### EXAMPLE 3

The following experiments were conducted to determine the effect of plasma treatment on adhesive strength between PVA and silicone used in ophthalmic drug delivery devices.

PVA strips were cut from precured PVA film, and then the strips were placed in an oven for three hours at 150° C. to further cure the strips. Silicone strips made of PDMS were provided. The PVA strips simulate a suture tab and the silicone strips simulate a silicone holder. Some of the silicone strips holders were extracted in isopropanol (IPA), while some holders were not extracted. Some of the holders were plasma treated in an oxygen environment, while some holders were not plasma treated. The silicone strips were cut to a size of 1.93×3.81 mm. PVA strips were glued to one side of the silicone holders using a 10% PVA solution, left overnight, and then cured in an oven for five hours at 135° C. Acetate strips were glued to the other side of the silicone holders using an epoxy resin, and left overnight. The assemblies were then hydrated in borate buffered saline solution overnight. Mechanical testing was conducted on individual assemblies by gripping a PVA strip with one gripping clamp and an acetate strip with another gripping clamp, under the following conditions: 6.35 mm/min, 15.95 mm gauge length, and 2,500 g load cell. The results are summarized in Table 3, noting that the reported measurement is the load (g) reached at breakage and seven samples were used for each treatment.

TABLE 3

| Treatment | | Average Breakage | |
|---|---|---|---|
| Plasma | Extraction | (std deviation) | Note |
| None | None | 26.46 (10.66) | 3/7 fell |
| | | | apart during hydration |
| | | | 4 broke at PVA/ |
| | | | silicone bond |
| None | IPA | 21.73 (11.21) | 3/7 fell |
| | | | apart during hydration |
| | | | 2 broke at PVA/ |
| | | | silicone bond |
| | | | 2 broke at acetate/ |
| | | | silicone bond |
| O₂ | IPA | 197.07 (64.47) | 2/7 fell |
| | | | apart during hydration |
| | | | 5 broke at acetate/ |
| | | | silicone bond |

FIG. 3 illustrates another embodiment of this invention. In this embodiment, inner drug core 2 may have the form of a tablet, similar to the previous embodiment, including a mixture of active agent 3 and a permeable matrix material 4 such as PVA. Holder 6 may is made of an impermeable material, such as silicone, and in this embodiment, has the form of a tube with impermeable inserts 16, 17 at the ends of the tube. The opening in insert 17 forms the passageway 7 for passage of the active agent outside the device. Tab 10 may be made of PVA, and is attached to holder 6 with a permeable coating 18, made of a material such as PVA. In this embodiment, holder 6 is plasma treated to improve its wettability and the adherence of the PVA thereto. Preferably, holder 6 is extracted prior to plasma treatment.

FIG. 4 illustrates another embodiment of this invention. In this embodiment, inner drug core 2 may have the form of a tablet, similar to the previous embodiments, including a mixture of active agent 3 and a permeable matrix material 4 such as PVA. Holder 6 may is made of an impermeable material, such as silicone, and in this embodiment, has the form of a tube with an impermeable insert 16 added after the inner drug core tablet is placed in the holder. The opening in holder 6 forms the passageway 7 for passage of the active agent outside the device. In this embodiment, tab 10 is integrally formed, for example by molding, with outer permeable layer 20. Tab 10 may be made of PVA, and in this embodiment, tab 10 circumferentially surrounds the entire device. In this embodiment, holder 6 is plasma treated to improve its wettability and the adherence of the PVA thereto, and preferably, holder 6 is extracted prior to plasma treatment.

It will be appreciated the dimensions of the device can vary with the size of the device, the size of the inner drug core, and the holder that surrounds the core or reservoir. The physical size of the device should be selected so that it does not interfere with physiological functions at the implantation site of the mammalian organism. The targeted disease state, type of mammalian organism, location of administration, and agents or agent administered are among the factors which would effect the desired size of the sustained release drug delivery device. However, because the device is intended for placement in the eye, the device is relatively small in size. Generally, it is preferred that the device, excluding the suture tab, has a maximum height, width and length each no greater than 10 mm, more preferably no greater than 5 mm, and most preferably no greater than 3 mm.

The examples and illustrated embodiments demonstrate some of the sustained release drug delivery device designs for the present invention. However, it is to be understood that these examples are for illustrative purposes only and do not purport to be wholly definitive as to the conditions and scope. While the invention has been described in connection with various preferred embodiments, numerous variations will be apparent to a person of ordinary skill in the art given the present description, without departing from the spirit of the invention and the scope of the appended claims.

1. A drug delivery device, comprising:
a drug core comprising a pharmaceutically active agent;
a holder that holds the drug core, the holder being made of a material impermeable to passage of the active agent; and
a tab for attaching the device to eye tissue,
wherein a surface of the holder is plasma treated and adhered to at least one of the drug core and the tab.

2. The device of claim 1, wherein the drug core comprises a mixture of the active agent and a matrix material permeable to said active agent.

3. The device of claim 2, wherein the matrix material comprises polyvinyl alcohol.

4. The device of claim 1, wherein the holder partially surrounds the drug core and includes a passageway for the active agent therethrough.

5. The device of claim 1, wherein the holder comprises a cylinder that surrounds the drug core, and an end of the cylinder includes an opening for passage of the active agent therethrough.

6. The device of claim 1, wherein the impermeable material comprises silicone.

7. The device of claim 1, wherein the drug core is cylindrical.

8. The device of claim 1, wherein the holder completely surrounds the drug core and includes an opening therein for passage of the active agent therethrough.

9. The device of claim 1, wherein the drug core is coated with a material permeable to said active agent.

10. The device of claim 1, wherein the impermeable material comprises silicone that is extracted with a solvent prior to plasma treatment.

11. The device of claim 1, wherein the tab is adhered to the plasma treated surface of the holder.

12. The device of claim 11, wherein the tab is adhered to the core with an adhesive.

13. The device of claim 1, comprising a mixture of pharmaceutically active agents.

14. A drug delivery device, comprising:
a drug core comprising a pharmaceutically active agent; and
a holder that holds the drug core, the holder being made of a material impermeable to passage of the active agent,
wherein a surface of the holder contacting the drug core is plasma treated and adhered to the drug core.

15. A method of making a drug delivery device, comprising:
providing a holder made of a material impermeable to passage of a pharmaceutically active agent and plasma treating a surface of the holder;
inserting in the holder a drug core comprising the active agent; and
adhering to the plasma treated surface a tab for attaching the device to eye tissue.

16. The method of claim 15, wherein the drug core comprises a mixture of the active agent and a matrix material permeable to said active agent.

17. The method of claim 16, wherein the matrix material comprises polyvinyl alcohol.

18. The method of claim 15, wherein the holder partially surrounds the drug core and includes a passageway for the active agent therethrough.

19. The method of claim 15, wherein the holder comprises a cylinder that surrounds the drug core, and an end of the cylinder includes an opening for passage of the active agent therethrough.

20. The method of claim 15, wherein the impermeable material comprises silicone.

21. The method of claim 15, wherein the drug core is cylindrical.

22. The method of claim 15, wherein the holder completely surrounds the drug core and includes an opening therein for passage of the active agent therethrough.

23. The method of claim 15, wherein the drug core is coated with a material permeable to said active agent.

24. The method of claim 15, further comprising extracting the holder with a solvent prior to plasma treatment.

25. The method of claim 15, wherein the tab is adhered to the plasma treated surface of the holder.

26. The method of claim 25, wherein the tab is adhered to the core with an adhesive.

27. The method of claim 15, wherein the core comprises a mixture of pharmaceutically active agents.

28. A method of making a drug delivery device, comprising:
providing a drug core comprising a pharmaceutically active agent;
providing a holder made of a material impermeable to passage of the active and plasma treating a surface of the holder; and
inserting the drug core in the holder, and adhering the drug core to the plasma treated surface of the holder.

29. A method comprising:
providing a drug delivery device, comprising:
a drug core comprising a pharmaceutically active agent;
a holder that holds the drug core, the holder being made of a material impermeable to passage of the active agent, and
a tab for attaching the device to eye tissue,
wherein a surface of the holder is plasma treated and adhered to at least one of the drug core and the tab; and
attaching the tab to eye tissue.

30. The method of claim 29, wherein the tab is attached by suturing.

31. The method of claim 29, wherein the device is implanted at the back of the eye.
